(19) United States
(12) Patent Application Publication
(10) Pub. No.: US 2020/0315831 A1
(43) Pub. Date: Oct. 8, 2020

(54) FOOT LIFT ORTHOSIS

(71) Applicant: OTTOBOCK SE & CO. KGAA, Duderstadt (DE)

(72) Inventor: Alexander HARDT, Asslar (DE)

(73) Assignee: OTTOBOCK SE & CO. KGAA, Duderstadt (DE)

(21) Appl. No.: 16/305,321

(22) PCT Filed: May 30, 2017
(86) PCT No.: PCT/EP2017/062976

§ 371 Date: Nov. 28, 2018

(30) Foreign Application Priority Data
May 31, 2016 (DE) 102016109963.5

(51) Int. Cl. A61F 5/01

(52) U.S. Cl. CPC A61F 5/0113

(57) ABSTRACT

A foot lift orthosis comprising at least one tensile element which is configured, when the foot lift orthosis has been attached, to extend from a forefoot region of a foot along an arch of the foot and to exert a first tensile force onto the forefoot region in the direction of a first bearing position above an upper ankle joint, and at least one second tensile element which is configured, when the foot lift orthosis has been attached, to exert a second tensile force onto the forefoot region in the direction of a second bearing position in a heel region of the foot.

The invention relates to a foot lift orthosis.

Foot drop, where the foot is in a “supination” position, is common following a stroke, for example, but can also have other causes. When foot drop occurs, the front of the foot can no longer be sufficiently lifted, and therefore the risk of tripping is significantly increased.

A number of different foot lift orthoses for counteracting this issue are known from the prior art. One of the foot lift orthoses having the simplest design consists of a cast that is positioned around the wearer's leg above the ankle, and which comprises straps or tension elements that are connected to the shoe, in particular to laces of a shoe. An upward force is exerted on the shoe as a result, which force therefore also lifts the foot. However, a disadvantage is that an orthosis of this type cannot be worn barefoot or with all types of shoe, and can easily been seen by others from the outside.

Alternatively, the tension element originating at the cast arranged above the ankle can also be arranged on a second cast which surrounds the foot in the frontal foot region. The tension element thus tensions an approximately diagonally extending tension connection between the wearers foot and leg, and therefore a foot lift orthosis of this type cannot be worn with a shoe. Moreover, the forefoot is compressed in the peripheral direction by the surrounding cast which applies the force to the foot, which is often to be avoided from a therapeutic point of view. Rather than lifting the whole foot, an upward force that is only exerted on the forefoot instead allows the foot to “flex,” resulting in the foot being stretched or even overstretched. This is not advantageous either.

Another type of foot lift orthosis comprises a stable sole element that extends over a smaller or larger part of the sole of the foot. Said element is made of carbon fiber composite, for example, and connected to a support device that is generally arranged on the patient's lower leg. The stable sole plate that extends along and underneath the foot lifts the foot, and thus also lifts the front of the foot. However, most of these foot lift orthoses require a surrounding shoe, and in particular cannot be worn barefoot because they can be easily seen from the outside. It is also not possible to achieve the feeling of being barefoot because the sole of the foot is largely covered by the sole element.

The problem addressed by the invention is therefore that of proposing a foot lift orthosis that can also be worn barefoot and is easy to clean, while nevertheless being cost-effective and simple to produce.

The invention solves the stated problem with a foot lift orthosis comprising at least a first tension element designed to extend from a frontal region of a foot along an instep of the foot and to exert, on the frontal foot region, a first tensile force toward a first support position above an upper ankle joint when the foot lift orthosis is being worn, and also comprising at least a second tension element designed to exert, on the frontal foot region, a second tensile force toward a second support position in a heel region of the foot when the foot lift orthosis is being worn.

The second tension element is advantageously designed to extend from the frontal foot region along a sole of the foot when the foot lift orthosis is being worn.

The foot lift orthosis according to the invention therefore has two tension elements, of which the first tension element extends over the instep and the second tension element preferably extends along the sole of the foot. One end of each of the two tension elements acts on the frontal foot region, for example in the region of the ball of the foot, and exerts a tensile force on said frontal foot region. The first tension element exerts a tensile force that is oriented toward the first support position, which is located above the upper ankle joint. The foot region is lifted as a result, and the function of the foot lift orthosis is therefore ensured. The second tension element simultaneously exerts a second tensile force that extends approximately or completely in parallel with the sole of the foot and thus prevents the foot from flexing. Since both applied tensile forces act in the frontal foot region, said forces result in the foot being “compacted.” Both forces in combination therefore ensure that the foot is held by a force oriented toward the heel, such that in particular the plantar fascia, i.e. the aponeurosis of the foot, is tensed. Any adverse flexing of the foot by means of the upward forces acting on the forefoot, as is possibly known from the relevant prior art, is prevented in this way.

Since the first tension element extends along the instep and the second tension element preferably extends along the sole of the wearers foot, the orthosis is very discreet and can be used with and without a shoe, and in particular can also be used barefoot. Since the foot lift orthosis is in contact with almost the entire surface of the foot, it is not immediately noticeable provided that it is made of a transparent or skin-colored material, and can therefore be worn inconspicuously even barefoot.

The first tension element and the second tension element are advantageously interconnected in the frontal foot region, and are preferably integral with one another. This one-piece embodiment can be made of silicone, for example. However, it is possible in this case to design the two tension elements having different thicknesses or different widths and thus to produce tensile forces of different intensities, even when identical materials are used and the two tension elements are integral with one another. Of course, it is also possible to make one or both of the tension elements out of silicone and to interconnect said elements in the frontal foot region in a different way.

The second tension element preferably has an opening such that said element can be wrapped around the heel of the foot. In this case, putting on the foot lift orthosis is particularly easy in the region of the second tension element because the second tension element just needs to act in the frontal foot region, and the wearer's heel can then be guided through the opening in the second tension element. In this way, the heel acts as a counterweight for the second tensile force applied by the second tension element. Alternatively or additionally, it is possible to provide enclosing means for the foot that act as a counterweight for the second tensile force in this way. A tab or other grip element that further simplifies the process of putting on the orthosis can also be provided.

The first tension element advantageously has at least one opening such that said element can be wrapped around an ankle region. The first tension element extends around the wearer's leg once above the ankle, such that said leg acts as a counterweight for the first tensile force. In order to put on the foot lift orthosis, the foot is simply guided through said opening in the first tension element, a connection region between the first tension element and the second tension element is arranged in the frontal foot region and the heel is subsequently advantageously guided through the opening in the second tension element. Since it is not necessary to fasten any bands or straps or tie any shoelaces or cords, the foot lift orthosis can be put on in an easy and reproducible manner.

The first tension element preferably follows the extension of the plantar fascia. Particularly advantageously, only a small part of the sole of the foot is covered in this case, in particular the region of the ball of the foot, in order to give the wearer as far as possible the feeling of actually walking barefoot when the foot lift orthosis is worn directly on the foot, i.e. without socks, stockings or shoes.

In a particularly preferred embodiment, the foot lift orthosis comprises at least one supporting link that extends upward from the first support position and can be guided around the wearers lower leg. This occurs in particular in the posterior region of the wearer's leg. In this way, tensile force applied by the first tension element is thus deflected toward the posterior. When the foot lift orthosis is being worn, the supporting link extends upward on the wearers lower leg and is then guided around the lower leg. The tensile force applied by the first tension element can also be altered in this way, and in particular increased. The enclosing means around the lower leg, which are part of the supporting link, are advantageously designed such that they can be reversibly opened and closed. This can for example be achieved by means of a button or an interlocking connection element, e.g. via hook and loop closures. This significantly simplifies the process of putting on and taking off the foot lift orthosis.

At least one opening through which at least one toe of the foot extends when the orthosis is being worn is advantageously located between the first tension element and the second tension element. Of course, it is also possible to provide an opening through which more than one toe extends, and/or to provide at least one opening, and advantageously a plurality of openings, with one toe extending through each opening. This occurs in the connection region between the first tension element and the second tension element, which region can be designed as a separate component that is connected to the first tension element and to the second tension element, or as a transition region between the two tension elements. Needless to say, it is also conceivable for said region to be integral with the first tension element and with the second tension element such that the opening is provided in a silicone element, for example. The tensile force that can be applied by each of the two tension elements is applied in said transition region between the first tension element and the second tension element, such that the toes are secured in the opening through which at least one toe of the foot extends. Of course, it is also possible for an opening through which all the toes are guided to be located in said region such that the foot lift orthosis is only supported on the ball of the foot in the frontal foot region.

The foot lift orthosis preferably comprises at least one reinforcement element that can be fastened on the lower leg and extends along the instep such that the first tensile force that can be applied by the first tension element can be increased. The at least one reinforcement element is in particular a separate component, and can therefore easily be replaced by another reinforcement element. In this way, the total force that can be applied by the foot lift orthosis can be individually adjusted to the wearer's needs particularly easily, and if necessary can be adapted to changing situations during treatment, for example a progressive recovery. One end of the at least one reinforcement element can comprise a securing device by means of which said element can be secured to the wearer's lower leg. This too is achieved by enclosing means that can be detachably fastened using a closure element. The opposite end of the reinforcement element can be arranged on the first tension element by means of interlocking connection means, or in another way. This advantageously occurs as close as possible to the frontal region of the foot, such that a tensile force can also be exerted on said frontal foot region by means of the reinforcement element. It has shown to be advantageous when the reinforcement element is arranged on the wearer's lower leg in such a way that said element is also enclosed by the supporting link and the enclosing means of the lower leg thereof, in order to ensure that the foot lift orthosis is overall as discreet as possible.

The foot lift orthosis preferably comprises at least one stabilizing element that extends around the frontal foot region when the foot lift orthosis is being worn.

In an embodiment that is particularly simple to produce and particularly easy to clean, the foot lift orthosis is made of silicone and is advantageously formed in one piece. In this case, the entire foot lift orthosis consists of a single silicone element in which the required openings are advantageously located. When not being worn, said orthosis can be folded up so as to be particularly small such that it is simple to store, transport and put on, and is in particular impervious to salt water and easy to clean. A reinforcement element can also be designed as a separate component in this case. Said element is preferably produced from the same material as the rest of the foot lift orthosis.

If the foot lift orthosis is a separate element that is in particular made of silicone and produced from a single workpiece, it is advantageous for the foot lift orthosis to be individually adaptable. It goes without saying that this is also advantageous for foot lift orthoses formed in a plurality of pieces or other types of foot lift orthosis.

Said individual adaptability can for example be achieved by making it possible e.g. for openings or holes that are provided in the foot lift orthosis to be produced and cut into the foot lift orthosis individually. These can be, for example, cut-outs or openings through which the heel projects when the foot lift orthosis is being worn. A further opening, which can preferably be designed individually, is intended for the toes of the foot when the orthosis is being worn. The access opening through which the foot is guided when the foot lift orthosis is being put on can preferably also be customized in this way. The position and/or the size of at least one of said openings, but preferably of a plurality or all of said openings, can preferably be adjusted. Feet of different sizes, for example, can be accommodated in this way, e.g. because the spacing between the opening for the heel and the opening for the toes can be increased or decreased. In a preferred embodiment, the required openings are cut into the material of the foot lift orthosis using scissors or another cutting tool, for example. Marking lines which mark particular shoe sizes for which the orthosis is intended to be suitable, for example, can be provided on the foot lift orthosis for this purpose. An orthopedic technician or the wearer of the orthosis then simply has to cut the desired opening into the material of the foot lift orthosis along the line that is suitable for them, and in this way can create a foot lift orthosis that is individually produced for them. In particular, unique physical features and the individual condition of the relevant foot can be catered for in this way.

In addition to the size and particular physical characteristics of the relevant foot, it is also possible to accommodate the toes by allowing the size of the hole for the toes to be varied, for example, and to thus design the foot lift orthosis to be as comfortable as possible. It is therefore possible to cut a plurality of holes into the material of the foot lift orthosis with each hole being for one toe, or to cut one hole for a plurality of toes. Combinations of these hole configurations can also be used, of course.

Alternatively, it is also possible for the foot lift orthosis to comprise a stocking made of a textile material on which the first tension element and the second tension element are arranged, preferably bonded or molded. Said stocking is preferably provided with an anti-slip coating on an inner side thereof that faces the foot. The coating can be applied over the whole surface or in regions, preferably in the heel region, the ankle region and/or the frontal foot region. A silicone coating is preferably used.

An embodiment of the present invention is described in more detail in the following with references to the attached drawings, in which:

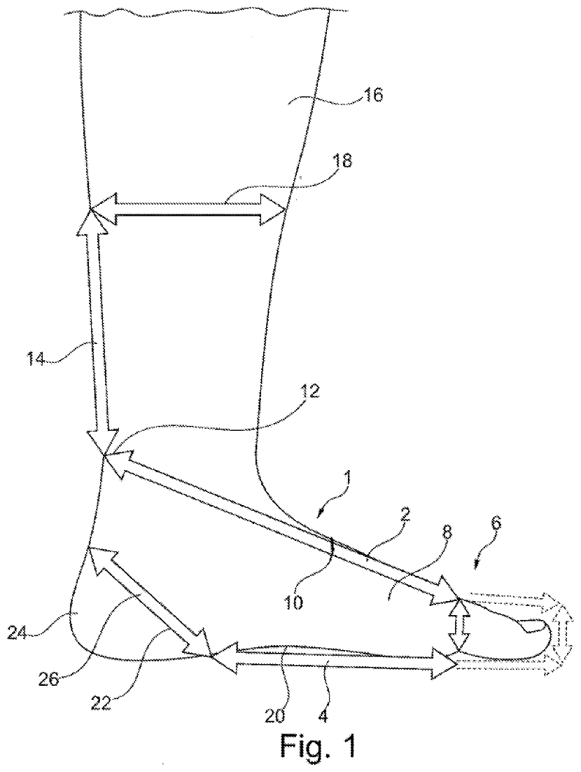

FIG. 1 is the schematic side view of a foot lift orthosis according to a first embodiment of the present invention,

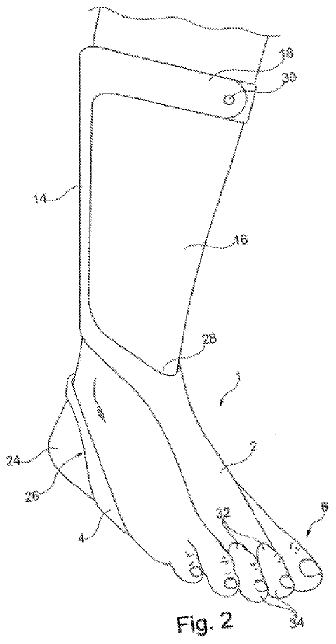

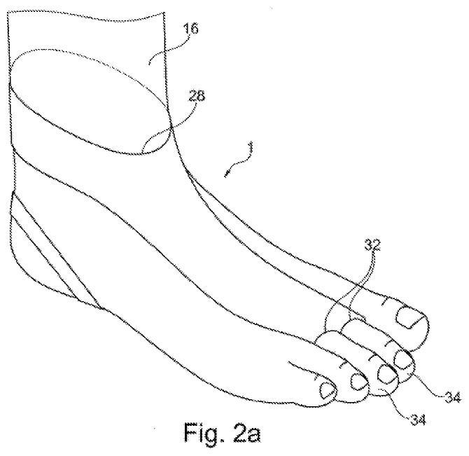

FIG. 2 is the schematic 3D view of a foot lift orthosis,

FIG. 2a is a slight modification of the embodiment shown in FIG. 2,

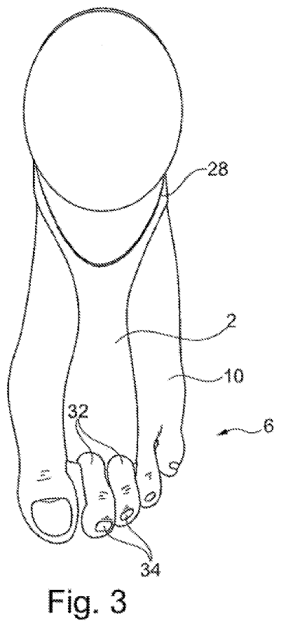

FIG. 3 is the schematic plan view of the foot lift orthosis from FIG. 2,

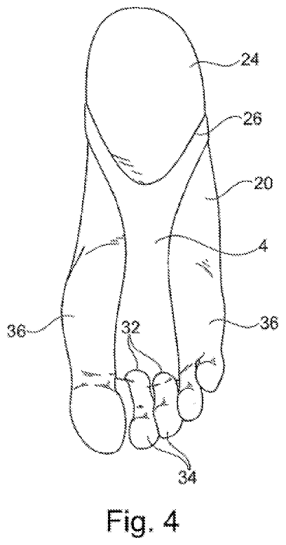

FIG. 4 is a view of the foot lift orthosis from below,

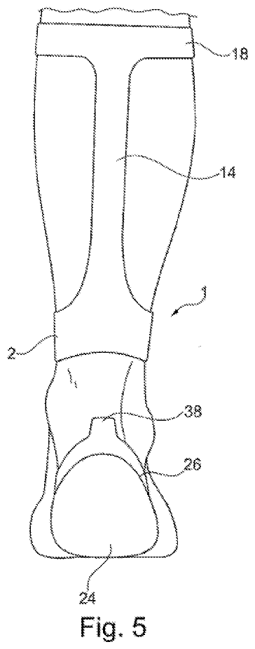

FIG. 5 is the schematic rear view of the foot lift orthosis,

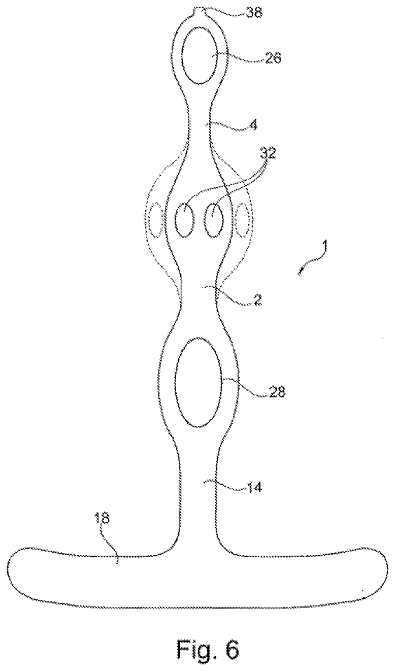

FIG. 6 is the schematic view of the foot lift orthosis when it is not being worn.

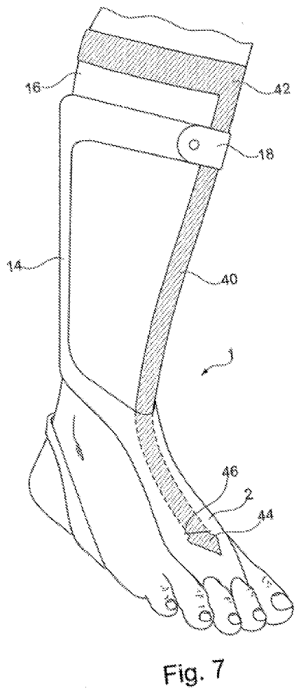

FIG. 7 is the schematic view of a foot lift orthosis according to another embodiment of the present invention,

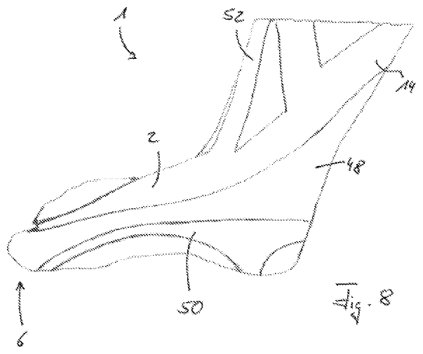

FIG. 8 is the schematic view of a foot lift orthosis according to another embodiment of the present invention,

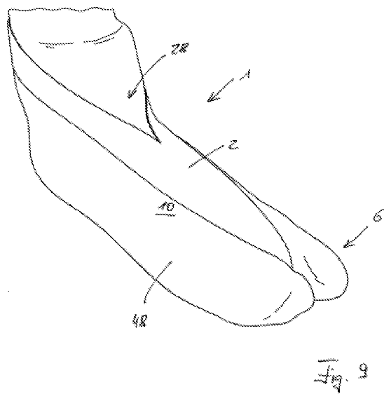

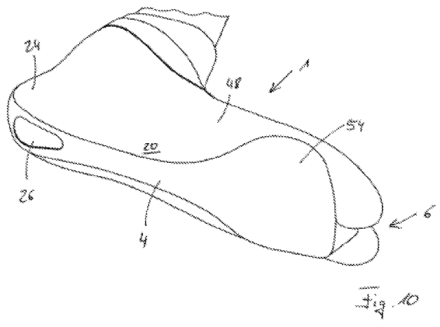

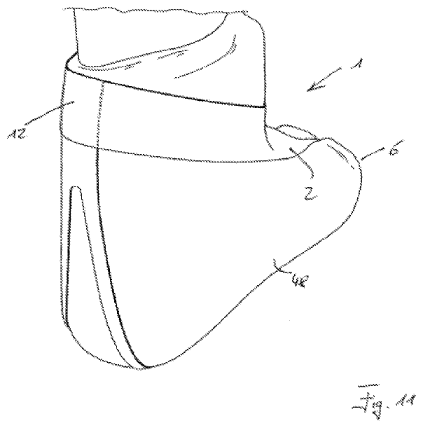

FIG. 9-11 are schematic views of a foot lift orthosis according to another embodiment of the present invention,

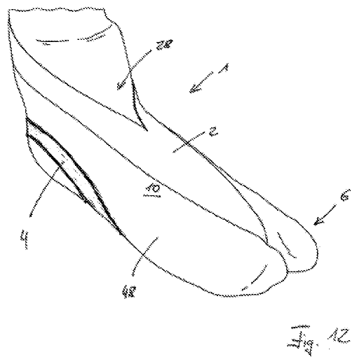

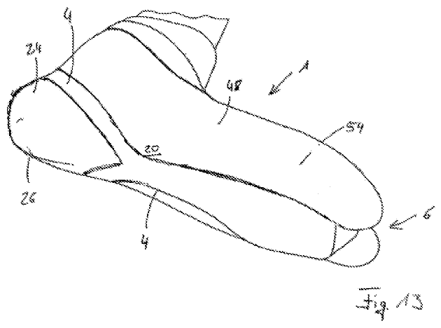

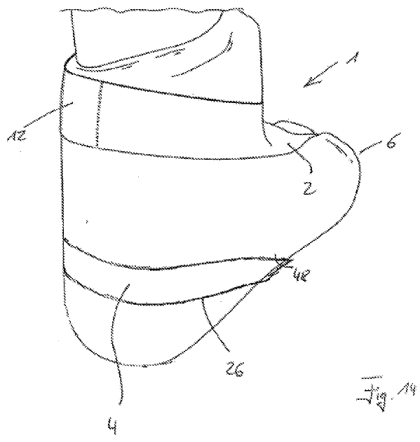

FIG. 12-14 show another embodiment, and

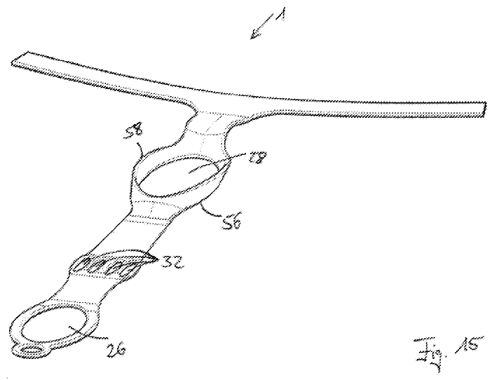

FIG. 15 is the schematic view of another embodiment.

FIG. 1 is the schematic view of a foot lift orthosis 1. It comprises a first tension element 2 and a second tension element 4, one end of each element acting on a frontal foot region 6 of a foot 8. The first tension element 2 extends from the frontal foot region 6 along an instep 10 to a first support position 12 which is located above the upper ankle joint. In this way, a first tensile force which extends along the first tension element 2 can be exerted on the frontal foot region 6. A supporting link 14 that extends upward along the lower leg 16, and comprises surrounding means 18 that can be wrapped around the lower leg 16, is attached to the posterior end of the first tension element 2. In this case, the surrounding means 18 preferably comprise a closure element (not shown in FIG. 1), such that the surrounding means 18 can be opened in order to put on and take off the foot lift orthosis.

The second tension element 4 that also acts in the frontal region 6 of the foot 8 extends along a sole 20 of the foot as far as a second support position 22 in the region of the heel 24 of the foot 8. The second tension element 4 has an opening 26 through which the heel 24 extends, and the first element 2 in the embodiment shown in FIG. 1 has an opening (not shown) through which the lower leg 16 extends.

FIG. 2 is a schematic three-dimensional view of the foot lift orthosis 1. The first tension element 2 can be seen, along with part of the second tension element 4, through the opening 26 of which the heel 24 extends. The opening 28 in the first tension element 2 through which the lower leg 16 extends can now be seen. The supporting link 14 and the surrounding means 18 attached thereto can be seen in the posterior region of the lower leg 16, which means now comprise a closure element 30 that is designed as a pin guided through a hole in the present example.

Two openings 32 are shown in the frontal foot region 6, a toe 34 extending through each of said openings.

FIG. 2a shows an embodiment of the foot lift orthosis 1 that corresponds substantially to the embodiment shown in FIG. 2. The lower leg 16 extends through the opening 28, whereas the toes 34 protrude through the openings 32. However, in contrast to the embodiment shown in FIG. 2, the foot lift orthosis 1 shown in FIG. 2a does not have a supporting link 14 and surrounding means 18 by means of which a further retaining position is achieved on the lower leg 16.

FIG. 3 is a plan view of the orthosis shown in FIG. 2. The first tension element 2 extends over the instep 10 from a frontal foot region 6 toward the ankle region, and has an opening 28 through which the leg is guided. The two openings 32 through each of which a toe 34 extends can be seen in the frontal foot region 6.

FIG. 4 is a view of the foot lift orthosis 1 from below. The sole 20 of the foot and the second tension element 4 can be seen, the heel 24 extending through the opening 26 in said element. A large portion of a ball 36 is not covered by the second tension element 4, such that the wearer of an orthosis can experience the feeling of being barefoot when wearing the orthosis 1 in the embodiment shown without socks and shoes. As in FIG. 3, two toes 34 extend through the openings 32.

FIG. 5 is a rear view of the foot lift orthosis 1. The heel 24 extends through the opening 26 in the second tension element 4. A tab 38 is located on said second tension element, which tab facilitates putting on the orthosis because the tab 38 can be gripped, and therefore the second tension element 4 can be tensioned and the heel 24 can be guided through the opening 26. As has already been explained, the supporting link 14 having the surrounding means 18 is attached to the second tension element 2.

FIG. 6 shows the foot lift orthosis 1 when it is not being worn. The foot lift orthosis 1 as shown in FIG. 6 is formed in one piece and is advantageously made of silicone. This embodiment is particularly easy to clean, simple to produce and is in particular waterproof, in particular also in the case of sea water, i.e. salt water. It can clearly be seen that the openings 32 for the toes are located between the first tension element 2 and the second tension element 4. In an alternative embodiment of the foot lift orthosis 1, which is represented by dashed lines in FIG. 6, the number of openings 32 is increased to 4 such that four toes can be guided through said openings 32, one toe being guided through each opening. Of course, it is also possible to design the openings 32 such that more than one toe can be guided through each opening.

The opening 26 in the second tension element 4, through which the heel 24 can be guided, is located at the end of said element that is opposite the openings 32. The opening 28 is located at the end of the first tension element 2 that is opposite the openings 32. In this case, in order to put on the orthosis, the foot is guided through the opening 28, and the region between the two tension elements 2, 4 in which the openings 32 are located is subsequently arranged on the toes and in the frontal region 6 of the foot. The first tension element 2 is tensioned as a result and already exerts a tensile force on the forefoot 6. The second tension element 4 can then be tensioned along the sole of the foot, and the heel 24 can be guided through the opening 26. The tab 38 can be gripped for this purpose, making it significantly easier to put on the foot lift orthosis 1. In addition, the supporting link 14 is also arranged along the lower leg and the enclosing means 18 are closed in order to put on the orthosis.

FIG. 7 shows the foot lift orthosis 1 according to another embodiment of the present invention. In addition to the first tension element 2 and the supporting link 14 that comprises the enclosing means 18 and is attached to said element, the foot lift orthosis shown in FIG. 7 also has a reinforcement element 40 that can be fastened to the wearer's lower leg 16 in the upper region by means of further enclosing means 42. In the embodiment shown, the lower region of the reinforcement element 40 is fastened to the first tension element 2 by means of an arrowhead-shaped interlocking connection element 44. For this purpose, the interlocking connection element 44 is guided through a slot 46 provided in the first tension element 2. The reinforcement element is also advantageously resilient. However, an additional tensile force that increases the tensile force of the first tension element 2 can also be applied to the frontal foot region by means of a non-resilient reinforcement element 40.

The particular sectional shape, shown in particular in FIG. 6, of the foot lift orthosis that is preferably formed in one piece allows the orthosis to be put on easily. The second tension element 4 that follows the plantar fascia, and the opening 26 that provides an annular support around the heel 24, provide a counter-tension to the dorsal tensile stress. i.e. the first tensile force that is applied by the first tension element 2. Said dorsal tensile stress, which in the embodiment shown extends through the toes 34 and then annularly above the upper ankle joint, i.e. the first support position 12, allows the foot to be lifted. Different strengths, different material thicknesses and widths, or other changes to the parameters make it possible for tensile forces having different intensities to be exerted by the first tension element 2 and the second tension element 4. In order to prevent the individual elements from slipping on the foot and, if necessary, also to further reinforce the tensile stress direction, the supporting link 14 extends proximally on the posterior of the Achilles tendon and is for example secured below the calf at a spacing of approximately 15 to 20 cm by means of the surrounding means 18. This is also advantageously carried out entirely using a waterproof material that is in particular not corroded by salt water.

The closure of the surrounding means 18 remains in its place by means of the supporting link and cannot slide down in the direction of the talus.

FIG. 8 shows another design of the foot lift orthosis 1 according to another embodiment of the present invention. Various strips 50 made of the material of the foot lift orthosis 1 are applied, for example bonded or molded, to a stocking 48. The first tension element 2, which is guided around the toes in the frontal foot region 6, can be seen. In the example shown, the supporting link 14 is cruciform. Further stabilizing elements 52 are also provided. Designing said stabilizing elements as the stocking 48 largely prevents the foot lift orthosis 1 from slipping on the foot 8, such that the elements 14, 52 used for stabilization, together with other components which are not shown, can be designed differently.

FIG. 9 to 11 show a particularly simple embodiment of the foot lift orthosis 1. The stocking 48 to which the material of the foot lift orthosis 1 is applied can be seen. The first tension element 2 extends over the instep 10 as far as the frontal foot region 6. The foot is guided through the opening 28, and the first support position 12 is formed in the rear region of the foot (in the region of the Achilles tendon in the embodiment shown). In the region of the sole 20 of the foot, the second tension element 4 extends from the frontal foot region 6 as far as the heel 24, where the opening 26 is located. A widening 54 in the material of the foot lift orthosis 1, which can for example be silicone, is located in the region of the ball of the foot. It is therefore ensured that the orthosis material is distributed as evenly as possible, in particular in this region in which a high load acts on the foot when said orthosis is being worn, and therefore uncomfortable or painful pressure points can be avoided.

FIG. 12 to 14 show an embodiment of the foot lift orthosis 1 that corresponds to the embodiment that has the stocking 48 shown in FIG. 9 to 11. The first tension element 2 that extends over the instep 10 can also be seen here. However, the embodiment shown in FIG. 12 to 14 also comprises a second tension element 4 that extends around the heel 24, which protrudes through the opening 26 that is formed.

FIG. 15 shows another embodiment of the foot lift orthosis 1. This embodiment also comprises the opening 26 through which the heel protrudes when the orthosis is being worn. Four openings 32 for the toes and the opening 28 through which the foot is guided when the orthosis is being put on can also be seen. The main difference with respect to the embodiment shown in FIG. 6 is the design of the foot lift orthosis 1 in the region of the opening 28. Rather than being completely flat, said opening has a three-dimensional contour having an upper edge 58 that is raised with respect to a lower edge 56. The wall extending between said edges is adapted to the geometry of the leg, which passes through the opening 28 when the foot lift orthosis 1 is being worn.

### LIST OF REFERENCE SIGNS

| | |
|---|---|
| 1 | foot lift orthosis |
| 2 | first tension element |
| 4 | second tension element |
| 6 | frontal foot region |
| 8 | foot |
| 10 | instep |
| 12 | first support position |
| 14 | supporting link |
| 16 | lower leg |
| 18 | surrounding means |
| 20 | sole of the foot |
| 22 | second support position |
| 24 | heel |
| 26 | opening |
| 28 | opening |
| 30 | closure element |
| 32 | opening |
| 34 | toe |
| 36 | ball |
| 38 | tab |
| 40 | reinforcement element |
| 42 | enclosing means |
| 44 | interlocking connection element |
| 46 | slot |
| 48 | stocking |
| 50 | strip |
| 52 | stabilizing element |
| 54 | widening |
| 56 | lower edge |
| 58 | upper edge |

1. A foot lift orthosis comprising:
at least a first tension element configured to extend from a frontal region of a foot along an instep of the foot when the foot lift orthosis is being worn, and configured to exert, on the frontal foot region, a first tensile force toward a first support position above an upper ankle joint; and
at least a second tension element configured to exert, on the frontal foot region, a second tensile force toward a second support position in a heel region of the foot when the foot lift orthosis is being worn.

2. The foot lift orthosis according to claim 1, wherein the second tension element is configured to extend from the frontal foot region along a sole of the foot when the foot lift orthosis is being worn.

3. The foot lift orthosis according to claim 1, wherein the first tension element and the second tension element are interconnected in the frontal foot region and are integral with one another.

4. The foot lift orthosis according to claim 1, wherein the second tension element has an opening and is configured to be wrapped around the heel of the foot.

5. The foot lift orthosis according to claim 1, wherein the first tension element has at least one opening and is configured to be wrapped around the ankle region.

6. The foot lift orthosis according to claim 1, further comprising at least one supporting link that extends upward from the first support position and is configured to be guided around a lower leg of the user when the orthosis is being worn.

7. The foot lift orthosis according to claim 1, further comprising at least one opening configured to receive at least one toe of the foot when the orthosis is being worn, the at least one opening being located between the first tension element and the second tension element.

8. The foot lift orthosis according to claim 6, further comprising at least one reinforcement element configured to be fastened on the lower leg and extend along the instep to permit increase of the first tensile force that can be applied by the first tension element.

9. The foot lift orthosis according to claim 6, further comprising at least one stabilizing element configured to extend around the frontal foot region when the orthosis is being worn.

10. The foot lift orthosis according to claim 1, wherein the foot lift orthosis is made of silicone and is formed in one piece.

11. The foot lift orthosis according to claim 1, wherein the foot lift orthosis comprises a stocking made of a textile material on which the first tension element and the second tension element are arranged.

12. A foot lift orthosis comprising:
a first tension element configured extend from a frontal region of a foot of a user along an instep of the foot when the foot lift orthosis, the first tension element being configured to exert a first tensile force on the frontal foot region in a direction toward a first support position above an upper ankle joint of the user; and
a second tension element configured to exert a second tensile force on the frontal foot region in a direction toward a second support position in a heel region of the foot.

13. The foot lift orthosis according to claim 12, wherein the second tension element is configured to extend from the frontal foot region along a sole of the foot when the foot lift orthosis is being worn.

14. The foot lift orthosis according to claim 12, wherein the first tension element and the second tension element are interconnected in the frontal foot region and are integral with one another.

15. The foot lift orthosis according to claim 12, wherein the second tension element has an opening and is configured to be wrapped around the heel of the foot.

16. The foot lift orthosis according to claim 12, wherein the first tension element has at least one opening and is configured to be wrapped around the ankle region.

17. The foot lift orthosis according to claim 16, further comprising at least one supporting link that extends upward from the first support position and is configured to be guided around a lower leg of the user when the orthosis is being worn.

18. The foot lift orthosis according to claim 12, further comprising at least one opening configured to receive at least one toe of the foot when the orthosis is being worn, the at least one opening being located between the first tension element and the second tension element.

19. The foot lift orthosis according to claim 17, further comprising at least one reinforcement element configured to be fastened on the lower leg and extend along the instep to permit increase of the first tensile force that can be applied by the first tension element.

20. The foot lift orthosis according to claim 12, further comprising at least one stabilizing element configured to extend around the frontal foot region when the orthosis is being worn.
